(19) United States
(12) Patent Application Publication
(10) Pub. No.: US 2010/0285054 A1
(43) Pub. Date: Nov. 11, 2010

(54) Antimicrobial Composition Comprising Fungal Extract, Process for Producing Fungal Extract and Method for Protecting Organisms

(76) Inventors: Helenires Queiroz De Souza, Manaus-AM (BR); Luiz Antonio De Oliveira, Manaus-AM (BR)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/810,380

(22) PCT Filed: Dec. 28, 2007
(86) PCT No.: PCT/BR07/00370

§ 371 Date: Jun. 24, 2010

(51) Int. Cl. A61K 36/07; A01N 65/00; A61P 31/04; A01P 15/00

(52) U.S. Cl. 424/195.15

(57) ABSTRACT

The present invention is directed to a fungal extract comprising antimicrobial activity. More specifically, the fungus used belongs to the family Polyporaceae, especially genus Trametes, namely Trametes sp. 11 E4. Specifically, the extract demonstrated activity against bacteria such as R. sonalacearum, E. coli, S. aureus, B. cereus and S. anatum. It also may be used in compositions, such as pest control compositions, which would be useful in agriculture, i.e. treating bacterial wilt and medicine.

### FIELD OF THE INVENTION

The present invention is directed to a fungal extract comprising antimicrobial activity. More specifically, the fungus used belongs to the family Polyporaceae, especially genus Trametes, namely Trametes sp. 11 E4.

Specifically, the extract demonstrated activity against bacteria such as R. sonalacearum, E. coli, S. aureus, B. cereus and S. anatum. It also may be used in compositions, such as pest control compositions, which would be useful in agriculture, i.e. treating bacterial wilt and medicine.

### BACKGROUND OF THE INVENTION

Plants are subject to attack by a great number of pathogens. These pathogens can be, for example, bacteria, fungi, or nematodes. Pesticidal compounds have long been used to increase yields and extend agricultural production capabilities into new areas. They have also been extremely important tools for ameliorating season-to-season differences in yield and quality caused by weather-driven variations in disease pressure.

The future role of pesticides in agriculture is increasingly threatened by several factors including the development of pest resistance, increasing concerns about food safety, and environmental accumulation of toxic compounds. As older pesticides are removed from the market due to regulatory changes, and new pesticides are becoming increasingly expensive to register, there is an increasing need to find ways to more wisely use the remaining, safest pesticides. This is particularly true for the many crop/disease combinations which do not represent large enough markets to pay for the cost of new compound registration. Wiser pesticide use will include ways to reduce application rates (and thus potential residues), finding ways to extend registrations to new crops, and identifying new compositions and treatments to combat the development of pest resistance.

R. solanacearum is an important phytopathogen with worldwide distribution and with a big number of hosts, more than 200 species in 50 families. Some of the most important hosts include tomato (Lycopersicum esculentum Mill.), pepper (Capsicum frutescens L.), potato (Ipomoea sp.), tobacco (Nicotiana tabacum L.), banana (Musa sp.), peas (Pisum sativum L.), peanut (Arachis hypogaea L.), cashew (Anacardium occidentale L.), papaya (Carica papaya L.) and olive (Olea europaea L.)

Several strategies for pest control include resistant host plants, trangenic plants, soil correction and biological control. Biological agents used for controlling tomato bacterial wilt includes arbuscular mycorrhizae, non-virulent mutants of R. solanacearum, antagonistic bacteria genetically engineered and some naturally occurring antagonistic rhizobacteria such as Bacillus spp., Pseudomonas spp., Streptomyces spp.

Chemical pesticides have provided an effective method of control; however, the public has become concerned about the amount of residual chemicals which might be found in food, ground water and the environment. Stringent new restrictions on the use of chemicals and the elimination of some effective pesticides from the market place could limit economical and effective options for controlling pests. In addition, the regular use of chemical toxins to control unwanted organisms can select for resistant strains.

Moura et al. (1998) evaluated the antagonistic potential of 190 actinomycetes isolates, obtained from different soils, rhizosphere, rhizoplane and internal tissues from healthy plants against isolates of R. solanacearum. Tomato seeds were treated with actinomycetes propagules under stirring for 10 minutes, and grown in pathogen infested soil. Eighteen isolates provided a 100% control.

Document WO 2006/065985 discloses compositions for fighting bacterial wilt in crops by using an extract of Yucca plants. The present invention differs from this prior art by using a fungal extract rather than a plant extract to control bacterial wilt.

Document EP 935918 discloses compositions of 3-(3-indolyl)-butanoic acid capable of selectively suppressing the growth of Ralstonia solanacearum in crops. The present invention differs from this prior art by using a fungal extract rather than a synthesized substance to control bacterial wilt.

Document EP 1 762 613 discloses insertion elements that have the ability of recombining with the genetic material of R. solanacearum, preventing further infection from this bacteria in crops. The present invention differs from this prior art by using a fungal extract rather than an insertion element to control bacterial wilt.

Document WO 2006/06133708 discloses polysaccharides obtained from several fungi species containing biological activity such as antineoplasic. One suitable genus is Trametes sp. The present invention differs from this prior art by using an extract and filtered cultures against bacterial wilt, and not cancer. Further, the present invention comprises a step of polysaccharides precipitation.

Document US 2007/093387 discloses a composition comprising a laccase with antimicrobial/antiviral properties, wherein said laccase can be obtained from several microorganisms, such as fungus from Trametes genus. The present invention differs from this prior art in that it uses extracts rather than isolated enzymes as antimicrobial agents.

Therefore, it can be seen from the available prior art that the use of Trametes sp. extracts as antimicrobial, specially in controlling bacterial wilt, as well as a process for producing this extract has never been disclosed or suggested.

### OBJECTIVE OF THE INVENTION

It is an objective of the invention an antimicrobial composition comprising:

a) an extract from a fungus belonging to Polyporaceae family; and

b) an acceptable vehicle.

It is a further objective of the invention a process for production an extract of a fungus belonging to Polyporaceae family comprising the steps of:
a) growing in a liquid medium a fungus belonging to Polyporaceae family; and
b) removing the mycelia from the liquid medium.

It is a further objective of the invention a method for protecting organisms from pathogenic microorganisms infection comprising the steps of:

a) contacting the plant with an antimicrobial composition comprising:
a. an extract from a fungus belonging to Polyporaceae family; and
b. an acceptable vehicle.

In a preferred embodiment, the process comprises the additional steps of further contacting the liquid medium with an adequate organic solvent.

In a preferred embodiment, the fungus is chosen from the genus Trametes. More specifically, the fungus is Trametes sp. 114E.

In a preferred embodiment, the extract is a filtered culture from a liquid medium growth. In addition to that, the adequate organic solvents can be hexane, dichloromethane, ethyl acetate and butanol.

### BRIEF DESCRIPTION OF THE FIGURES

FIG. 1 shows the procedure for inoculating the bacteria in solid medium.

FIG. 2 shows the scores given according to bacterial growth in Petri dishes with solid medium.

### DETAILED DESCRIPTION OF THE INVENTION

The following examples are intended solely to exemplify some embodiment of the many existent ways of performing the invention, and therefore are to be understood as not limiting the present invention.

Fungus Belonging to Polyporaceae Family

Suitable fungi according to the present invention include fungi belonging to the Polyporaceae family. Examples of fungi belonging to said family include, without limitation genera Ganoderma, Coriolus, Trametes and other related fungi.

In a preferred embodiment, the fungus is chosen from genus Trametes, namely Trametes sp. 11E4.

Pathogenic Microorganisms

According to the present invention, pathogenic microorganisms include, without limitation, bacteria, fungi, viruses and protozoa. The microorganism may also be a phytopathogenic microorganism.

Suitable bacteria include, without limitation, Gram-positive and Gram-negative bacteria belonging to the following genera: Staphylococcus, Ralstonia, Escherichia, Salmonella, Pseudomonas, Bacillus, Streptococcus and mixture thereof.

The composition of the present invention has a special selectivity for E. coli, S. aureus, B. cereus, S. anatum and R. solanacearum.

Organisms

Suitable organisms according to the invention include, without limitation, plants and animals.

Suitable animals include, without limitation mammals, such as humans.

Suitable plants include, without limitation, seeds, corms, bulbs, flowers, stems, leaves, exposed roots, and fruits of plants including, but not limited to, grapes, pears, apples, peaches, nectarines, grapefruit, cherries, apricots, lemons, limes, oranges, mangos, bananas, pineapple, and tangerines. Any plant which is susceptible to bacterial wilt disease caused by R. solanacearum is encompassed within the scope of the methods of the present invention. The plants which may be treated further include, but are not limited to, tomatoes, potatoes, bananas, peas, alfalfa, cabbage, clover, kale, lentil, soybean, sweet potato, radish, grape, cotton, sunflower, rape, chicory, chickpea, sorghum, onion, coconut, lily, sugarcane, cucumbers, squash, zucchini, eggplant, chili pepper, bell pepper, tobacco, groundnut, lettuce, cantaloupes, ginger, rice, corn, wheat, oats, barley, rye, millet, and other cereals, turfgrasses, and flower crops, including geraniums, other ornamental plants, nightshades, and almonds.

Antimicrobial Composition

The antimicrobial composition according to the present invention comprises:

a) an extract from a fungus belonging to Polyporaceae family; and

b) an acceptable vehicle.

The antimicrobial composition can be incorporated in cosmetic, pharmaceutical and/or agricultural compositions, in order to impart antimicrobial properties to the parent compositions.

In a preferred embodiment, the antimicrobial composition is added to an agricultural composition useful for crop protection, in order to protect crops from infections such as bacterial wilt.

### Example 1

### Microorganisms for Antimicrobial Activity

The pathogenic bacteria used in the assays were Escherichia coli, Staphylococcus aureus, Bacillus cereus, Salmonella anatum and Ralstonia solanacearum.

The R. solanacearum was originally isolated from several plants, such as tomatoes, cucumber, banana, eggplant, capsicum and moring a.

The bacteria were cultured in specific culture media:

R. solanacearum was purified in medium LPGA (5 g/L yeast extract, 5 g/L peptone, 5 g/L glucose 15 g/L agar, water, pH 7.0) and grown in 10 mL of LPG medium, at 28° C. for 48 hours.

E. coli, S. aureus, B. cereus and S. anatum: all were purified in nutrient agar (3 g/L meat extract, 10 g/L peptone, 5 g/L sodium chloride, 20 g/L agar, water, pH 7.0) and grown in 10 mL of liquid medium LB (10 g/L peptone, 5 g/L NaCl, 10 g/L yeast extract, pH 7.0), incubated at 37° C. for 24 hours with no agitation.

### Example 2

### Liquid Medium Culture

Fungus Trametes sp. 11E4, collected in Amazon, was subcultured for Petri dishes containing BDA (potato, 20 g/L dextrose, 15 g/L agar) and GPY (20 g/L glucose, 10 g/L peptone, 2 g/L yeast extract, 15 g/L agar, water; pH 6.6) media and incubated at 28° C. for 10 days. After the growing in BDA and GPY media, three mycelia plugs (10 mm) were transferred to 125 mL Erlenmeyers containing 50 mL of media BD (potato and 20 g/L dextrose) and GPY.

These Erlenmeyers were incubated in the dark at 28° C., by 30 days. After, the samples were filtered in sterilized paper in order to separate the mycelium. The filtered culture was stored at 4° C. prior to use.

### Example 3

### Growth Curve in Liquid Medium

The fungus was previously cultured in solid GPY (20 g/L glucose, 10 g/L peptone, 2 g/L yeast extract, 15 g/L agar, water; pH 6.6) and incubated at 28° C. for 10 days.

After the solid GPY culture, three mycelia dishes were inoculated into 16 Erlenmeyer flasks containing 50 mL of liquid medium. These were incubated at 28° C. during 8 weeks, at rest.

By the end of each incubation week, two samples were collected and filtered for mycelium separation. The culture filtered was stored at 4° C. prior to use. The mycelium from the liquid culture was weighed when still fresh and then put on Petri dishes with Aluminum foil and dried at 90° C. until constant weight.

### Example 4

### Evaluation of Antimicrobial Production During Mycelia Growth of Trametes sp.

The antimicrobial production was evaluated during the eight weeks of culture.

The lineage R. solanacearum V55 was chosen as test microorganism. The bacterium was cultured in liquid LPG, under stirring, for 24 hours. Then several and sequential dilutions of the bacterium culture were performed in MgSO₄ 2 g/L, varying from 10⁻¹ to 10⁻⁷.

Samples from the bacterial suspension (1 mL containing 10⁵ bacteria) plus a volume of the fungus filtered culture were put into test tubes. As a control, a tube with MgSO₄ 2 g/L was used, and all tubes were incubated at 30° C. for 24 hours. The table below gives details of each test tube.

TABLE 1
Treatments for antimicrobial evaluation during 8 week culture
of Trametes sp. 11E4 against R. solanacearum V55.

| Treatment | Constituent |
|---|---|
| A | R. solanacearum V55 (10⁵) + 1 mL of filtered culture from |
| | 1^{st} week |
| B | R. solanacearum V55 (10⁵) + 1 mL of filtered culture from |
| | 2^{nd} week |
| C | R. solanacearum V55 (10⁵) + 1 mL of filtered culture from |
| | 3^{rd} week |
| D | R. solanacearum V55 (10⁵) + 1 mL of filtered culture from |
| | 4^{th} week |
| E | R. solanacearum V55 (10⁵) + 1 mL of filtered culture from |
| | 5^{th} week |
| F | R. solanacearum V55 (10⁵) + 1 mL of filtered culture from |
| | 6^{th} week |
| G | R. solanacearum V55 (10⁵) + 1 mL of filtered culture from |
| | 7^{th} week |
| H | R. solanacearum V55 (10⁵) + 1 mL of filtered culture from |
| | 8^{th} week |
| Control | R. solanacearum V55 (10⁵) + 1 mL of 2% solution of MgSO₄ |

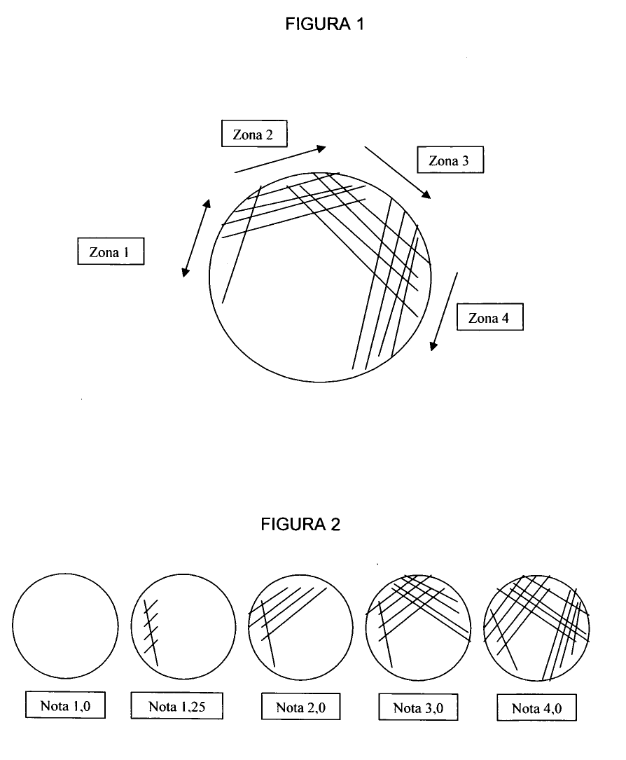

A sample of each test tube was removed with a platinum-loop and drawn into LPGA Petri dishes, according to FIG. 1. This technique consists of a phosphate solubilizing bacteria (BSF) growth scoring system. Intermediary grades are gives, subdivided in 0.25, i.e. 1.00, 1.25, 1.50, 1.75, 2.00 till 4.00, with increasingly precision, according to FIG. 2.

Bacteria with a grade higher than 3.06 was considered as tolerant.

The plates were incubated for 24 hours and then given the score for the growth. These scores were converted to % of bacterial growth, where a score of 4.0 corresponds of 100% growth.

Considering the antimicrobial potential of Trametes sp., from its growth curve it can be seen that the production of the antimicrobial compound began in the first week of culture.

### Example 5

### Evaluation of Antimicrobial Effect of the Filtered Culture in Different Dilutions

The fungal filtered culture, grown during 30 days, was diluted in sequential dilutions of a solution of MgSO₄ 2 g/L, from 10⁻¹ to 10⁻⁶.

Samples from the bacterial suspension plus a volume of: a) MgSO₄ 2 g/L; b) crude filtered culture; and c) diluted filtered culture, and all tubes were incubated at 30° C. for 24 hours. The table below gives details of each test tube.

TABLE 2
Treatments for antimicrobial evaluation of filtered culture
of Trametes sp. 11E4 against R. solanacearum.

| Treatment | | Constituents |
|---|---|---|
| A | 1 | 2 mL bacteria suspension 10⁶ + 2 mL of 2% solution |
| | | of MgSO₄ |
| | 2 | 2 mL bacteria suspension 10⁵ + 2 mL of 2% solution |
| | | of MgSO₄ |
| | 3 | 2 mL bacteria suspension 10⁴ + 2 mL of 2% solution |
| | | of MgSO₄ |
| | 4 | 2 mL bacteria suspension 10³ + 2 mL of 2% solution |
| | | of MgSO₄ |
| | 5 | 2 mL bacteria suspension 10² + 2 mL of 2% solution |
| | | of MgSO₄ |
| B | 6 | 2 mL bacteria suspension 10⁶ + 2 mL of filtered |
| | | culture |
| | 7 | 2 mL bacteria suspension 10⁵ + 2 mL of filtered |
| | | culture |
| | 8 | 2 mL bacteria suspension 10⁴ + 2 mL of filtered |
| | | culture |
| | 9 | 2 mL bacteria suspension 10³ + 2 mL of filtered |
| | | culture |
| | 10 | 2 mL bacteria suspension 10² + 2 mL of filtered |
| | | culture |
| C | 11 | 2 mL bacteria suspension 10⁵ + 2 mL of filtered |
| | | culture 10⁻¹ |
| | 12 | 2 mL bacteria suspension 10⁵ + 2 mL of filtered |
| | | culture 10⁻² |
| | 13 | 2 mL bacteria suspension 10⁵ + 2 mL of filtered |
| | | culture 10⁻³ |
| | 14 | 2 mL bacteria suspension 10⁵ + 2 mL of filtered |
| | | culture 10⁻⁴ |
| | 15 | 2 mL bacteria suspension 10⁵ + 2 mL of filtered |
| | | culture 10⁻⁵ |
| | 16 | 2 mL bacteria suspension 10⁵ + 2 mL of filtered |
| | | culture 10⁻⁶ |

A sample of each test tube was removed with a platinum-loop and drawn into LPGA Petri dishes, according to the scoring technique described above.

The crude filtered culture produced by the fungus presented antimicrobial activity against E. coli, S. aureus, B. cereus and S. anatum. The assays with different suspensions of R. solanacearum (10⁶ to 10²) with crude filtered culture no bacterial growth was observed. The antibacterial effect persisted in filtered culture diluted up to 10 times.

### Example 6

### Polysaccharides Precipitation to Obtain Fraction with Metabolites

A 50 mL sample of the filtered culture was added to 150 mL cool (8° C.) acetone and put to rest at 4° C. for 24 hours for polysaccharides precipitation. After that, two phases were observed, a white phase at the bottom and a colorless at the top. The colorless phase, containing acetone plus the metabolites was transferred to a new container, and lyophilized.

### Example 7

### Detection of Antibacterial Metabolites by Diffusion

6 mm diameter paper-filter discs were wet with the filtered culture, ca. 10 μL, and placed over LPGA medium (for phytopathogenic bacteria) and LB medium (for pathogenic bacteria), both media containing bacterial samples spread in the surface.

The dishes were incubated at 4° C. for 4 hours, for metabolites diffusion in the medium. Then were incubated at 30° C. for 48 hours (for phytopathogenic bacteria) and at 37° C. for 24 hours (for pathogenic bacteria).

Antibacterial activity was determined by the measurement of the inhibition zone diameter (in mm) and the average by the number of repetitions.

### Example 8

### Detection of Antibacterial Metabolites by Cup-Plate Technique

Eleven lineages of R. solanacearum were tested. Bacteria was cultured in solid LPGA for about 48 to 72 hours and then grown in 50 mL of liquid LPG and incubated at 28° C. for 24 hours under stirring. Dilutions were made by removing 1 mL samples and adding 9 mL of sterilized saline solution of MgSO₄ 2 g/L.

1 mL of each dilution was transferred to sterilized Petri dishes with LPGA at 40° C. After solidification of the medium, cup-plate were made with 6 mm diameter test tubes.

Each well received 100 μL of the filtered fungal culture. The dishes were incubated at 28° C. for 24 hours and observed for bacterial inhibition halos, measured with a caliper.

### Example 9

### Partition of Filtered Culture

Partition tests were performed in 2 L separation flasks, containing 300 mL of filtered culture and 300 mL hexane. The flask was gently stirred and opened for liberation of vapors. The system was put at rest until phase separation was evident. The hexane phase was separated from the aqueous phase.

The aqueous phase was put in a flask containing 300 mL of dichloromethane. The flask was gently stirred and opened for liberation of vapors. The system was put at rest until phase separation was evident. The dichloromethane phase was separated from the aqueous phase.

The process was repeated with different solvents with increasing polarity.

The aqueous phase suffered two more extractions: one time with 300 mL of ethyl acetate and three times 300 mL of butanol each time.

All the extracts (hexane, dichloromethane, ethyl acetate and butanol) had its solvents evaporated under reduced pressure and adequate temperatures.

The residue formed after each evaporation was transferred to adequate containers and protected with aluminum foils.

The remaining aqueous phase was freezed and then lyophilized.

### Example 10

### Biological Assays with the Extracts from the Filtered Culture

The extracts from the filtered culture were biologically tested in order to verify which fraction contains the active principle against R. solanacearum detected into the filtered culture.

The lineage R. solanacearum V55 was cultured in LPGA and 28° C. for 24 hours. Then it was transferred to a flask containing 50 mL of LPG, which was incubated for 24 hours at 28° C. under stirring.

50 μL of bacteria cell suspension (10⁸ UFC/mL—Colony Forming Unity) were inoculated in the center of a LPGA plate with a platinum loop through the spread plate technique.

6 mm diameter paper-filter discs were wet with 10 μL of the extracts and dried for an hour and a half until solvent evaporation. Then they were placed over the LPGA plates, which were incubates at 28° C. for 24 hours and observed for bacterial inhibition halos, measured with a digital caliper.

The controls used were discs with water, methanol, dichloromethane and dry paper-filter disc.

The crude and semi-purified extracts presented antibacterial activity against R. solanacearum. The inhibition halo was greater for the hexanic fraction (10.7 mm diameter) when compared with the dichloromethanic fraction (7.4 mm diameter).

### Example 11

### Extracts Chemical Constituents Analysis by Comparative Thin Layer Chromatography (Comparative TLC)

The extracts obtained from the partition experiments were analyzed by comparative thin layer chromatography. For each extract the suitable solvent for its solubilization was determined. The process begins with low polarity solvents until a clean extract with no particles in suspension is obtained.

The samples were applied in silica gel GF₂₅₄ Merck chromatopapers (5×3 cm) and tested different solvents for choosing an adequate elution system. The plates were observed under UV lamp (254 and 366 nm) and then revealed with sulfuric vanillin and heating (110° C.) until the observation of color.

The results revealed that terpenoids and steroids are present in these extracts.

### Example 12

### Infrared Spectroscopy Analysis

The samples were solubilized in adequate solvents and applied at the surface of a KBr pellet. The reading was performed in the range of from 4000 to 400 cm⁻¹ of infrared spectra.

The IR spectra of the filtered culture presented typical regions of a mixture of substances, suggesting, among other functions, alcohols and carbonyls. The spectra from the acetate and butanol phases were similar. The spectra from the hexane and dichloromethane phases were also similar, showing the presence of fatty acids and absence of proteins.

1. Antimicrobial composition comprising fungal extract characterized by the fact that it comprises:
a) from 1% w/w to 99% w/w of an extract from a fungus belonging to Polyporaceae family; and
b) an acceptable vehicle.

2. Antimicrobial composition, according to claim 1, characterized by the fact that the fungus is chosen from the genera Ganoderma, Coriolus and/or Trametes.

3. Antimicrobial composition, according to claim 2, characterized by the fact that the fungus is Trametes sp. 11 E4.

4. Antimicrobial composition, according to claim 1, characterized by the fact that the acceptable vehicle is chosen from the group comprising agricultural compositions, pharmaceutical compositions and mixtures thereof.

5. Process for producing fungal extract characterized by the fact that it comprises the steps of:
a) culture of at least one fungus belonging to Polyporaceae family in a suitable liquid medium; and
b) filtering the liquid medium.

6. Process, according to claim 5, characterized by the fact that the fungus is chosen from the genera Ganoderma, Coriolus and/or Trametes.

7. Process, according to claim 6, characterized by the fact that the fungus is Trametes sp. 11 E4.

8. Process, according to claim 5, characterized by the fact that the liquid medium comprises a mixture of potato, dextrose, agar, glucose, peptone, yeast extract, and water at pH 6.6.

9. Process, according to claim 5, characterized by the fact that the culture occurs in the dark at a temperature range of from 25° C. to 30° C.

10. Process, according to claim 5, characterized by the fact that the culture occurs for 7 days to 60 days.

11. Process, according to claim 10, characterized by the fact that the culture occurs for 30 days.

12. Process, according to claim 5, characterized by the fact that the fungus was previously grown in solid medium.

13. Process, according to claim 5, characterized by the fact that the filtered medium is stored at about 4° C. prior to use.

14. Process, according to claim 5, characterized by further comprising the step of extraction with an organic solvent.

15. Process, according to claim 14, characterized by the fact that the organic solvent is chosen from the group consisting of hexane, dichloromethane, ethyl acetate, butanol, acetone and mixtures thereof.

16. Process, according to claim 1, further comprising a step of lyophilization.

17. Method for protecting organisms comprising the step of applying to an organism a composition comprising:
a) from 1% w/w to 99% w/w of an extract from a fungus belonging to Polyporaceae family; and
b) an acceptable vehicle;

18. Method, according to claim 17, characterized by the fact that the fungus is chosen from the genera Ganoderma, Coriolus and/or Trametes.

19. Method, according to claim 18, characterized by the fact that the fungus is Trametes sp. 11E4.

20. Method, according to claim 17, characterized by the fact that the organism is chosen from the group consisting of plants and animals.

21. Method, according to claim 20, characterized by the fact that the animals are mammals.

22. Method, according to claim 20, characterized by the fact that the plants are chosen from the group consisting of seeds, corms, bulbs, flowers, stems, leaves, exposed roots, and fruits of grapes, pears, apples, peaches, nectarines, grapefruit, cherries, apricots, lemons, limes, oranges, mangos, bananas, pineapple, tangerines, tomatoes, potatoes, peas, alfalfa, cabbage, clover, kale, lentil, soybean, sweet potato, radish, cotton, sunflower, rape, chicory, chickpea, sorghum, onion, coconut, lily, sugarcane, cucumbers, squash, zucchini, eggplant, chili pepper, bell pepper, tobacco, groundnut, lettuce, cantaloupes, ginger, rice, corn, wheat, oats, barley, rye, millet, turfgrasses, geraniums, nightshades, almonds and mixtures thereof.

23. Method, according to claim 17, characterized by the fact that the composition is useful against bacteria belonging to the genera Staphylococcus, Ralstonia, Escherichia, Salmonella, Pseudomonas, Bacillus, Streptococcus and mixtures thereof.

24. Method, according to claim 23, characterized by the fact that the bacteria are chosen from the group consisting of E. coli, S. aureus, B. cereus, S. anatum, R. solanacearum and mixtures thereof.
